(19) United States
(12) Patent Application Publication
(10) Pub. No.: US 2025/0387624 A1
(43) Pub. Date: Dec. 25, 2025

(54) SYSTEMS AND METHODS OF IMPROVING SLEEP DISORDERED BREATHING

(71) Applicant: VANDERBILT UNIVERSITY, Nashville, TN (US)

(72) Inventor: David T. KENT, Nashville, TN (US)

(21) Appl. No.: 18/879,734

(22) PCT Filed: Jun. 30, 2023
(86) PCT No.: PCT/US2023/026741

§ 371 Date: Dec. 27, 2024

Related U.S. Application Data
(60) Provisional application No. 63/357,137, filed on Jun. 30, 2022.

(51) Int. Cl. A61N 1/36

(52) U.S. Cl. CPC A61N 1/3601; A61N 1/36003; A61N 1/36146

(57) ABSTRACT

Systems and methods for improving sleep disordered breathing are provided including delivering a stimulating neuromodulation signal to an ansa cervicalis that stimulates activation of the ansa cervicalis innervating one or more infrahyoid muscles. A blocking neuromodulation signal to the ansa cervicalis can be delivered that blocks activation of efferent fibers that innervate one or more suprahyoid muscles. The blocking neuromodulation signal can mitigate undesirable retrograde efferent activation of the one or more suprahyoid muscles.

### CROSS-REFERENCE TO RELATED APPLICATIONS

This application is a U.S. National Stage Application filed under 35 USC 371, claiming priority to Serial No. PCT/US2023/026741 filed on 30 Jun. 2023, which claims priority to U.S. Provisional Application No. 63/357,137 filed 30 Jun. 2022, the entireties of which are incorporated herein by reference.

### TECHNICAL FIELD

The present disclosure relates to systems and methods of improving sleep disordered breathing via neuromodulation by applying combination(s) of blocking neuromodulation signals and stimulating neuromodulation signals.

### BACKGROUND

Sleep disordered breathing (SDB) occurs when there is a partial or complete cessation of breathing that occurs many times throughout the night. Obstructive sleep apnea (OSA) is a type of SDB that involves cessation or significant decrease in airflow in the presence of breathing effort. It is the most common type of SDB and is characterized by recurrent episodes of upper airway collapse during sleep inducing repetitive pauses in breathing followed by reductions in blood oxygen saturation or neurologic arousal. The pathophysiology of OSA can involve factors such as craniofacial anatomy, airway collapsibility, and neuromuscular control of the upper airway dilator musculature. Electromyogram studies have shown that the tonic and phasic activity of the pharyngeal airway dilatory muscles (such as the genioglossus muscle) is progressively reduced from wakefulness to non-rapid eye movement to rapid eye movement.

Continuous positive airway pressure (CPAP) therapy is the frontline treatment for OSA. CPAP therapy utilizes machines, generally including a flow generator, tubing, and a mask designed to deliver a constant flow of air pressure to keep the airways continuously open in patients with OSA. However, the success of CPAP therapy is limited by compliance with reported rates ranging from 50% to 70%. Hypoglossal nerve stimulation (HNS) has now been established as an effective form of therapy for patients with obstructive sleep apnea (OSA) who are unable to tolerate positive airway pressure. This therapy works by protruding and stiffening the tongue muscle thereby dilating the pharyngeal airway. However, only a small subset of patients with OSA have anatomy suitable for hypoglossal nerve stimulation therapy, as many patients continue to suffer from airway collapse even with stimulation of hypoglossal nerve musculature.

### SUMMARY

Neuromodulation systems and method are provided to improve SBD by applying various combinations of stimulating neuromodulation signals and blocking neuromodulation signals to various neural target sites. In an aspect, a neuromodulation system to improving sleep disordered breathing can comprise a processor and a non-transitory computer readable medium storing executable instructions executable by the processor. Such executable instructions can include to direct delivery of a stimulating neuromodulation signal to an ansa cervicalis that stimulates activation of the ansa cervicalis innervating one or more infrahyoid muscles and to direct delivery of a blocking neuromodulation signal to the ansa cervicalis that blocks activation of efferent fibers that innervate one or more suprahyoid muscles, the blocking neuromodulation signal mitigating undesirable retrograde efferent activation of the one or more suprahyoid muscles.

In another aspect, a method to improve SBD in a patient suffering therefrom is provided. Such method can include delivering a stimulating neuromodulation signal to an ansa cervicalis that stimulates activation of the ansa cervicalis innervating one or more infrahyoid muscles. The method can further include delivering a blocking neuromodulation signal to the ansa cervicalis that blocks activation of efferent fibers that innervate one or more suprahyoid muscles, the blocking neuromodulation signal mitigating undesirable retrograde efferent activation of the one or more suprahyoid muscles.

### BRIEF DESCRIPTION OF THE DRAWINGS

FIG. 1 is a block diagram depicting illustrative components of a neuromodulation system according to an aspect of the present disclosure.

FIG. 2 is a block diagram depicting illustrative components of a neuromodulation system according to an aspect of the present disclosure.

FIG. 3 is a flow chart depicting illustrative steps of a method of improving SDB in a patient suffering therefrom.

### DETAILED DESCRIPTION

The present disclosure relates to neuromodulation systems and methods to improve SDB. In particular, systems and methods include delivering neuromodulation signals that activate neural target sites as well as delivering neuromodulation signals that block neural target sites. Blocking of neural target sites refers to suppressing neuronal actional potentials. For example, systems and methods are provided that direct stimulation of the ansa cervicalis, the hypoglossal nerve, the glossopharyngeal nerve, the pharyngeal plexus and/or branches thereof, or suitable combinations thereof to activate desired target muscles. In addition, systems and method are provided that direct blockage of undesirable motor targets of the ansa cervicalis, motor targets of the hypoglossal nerve, afferent sensory components of the glossopharyngeal nerve, or suitable combinations thereof. Non-limiting examples of SDBs are increased upper airway resistance including snoring, upper airway resistance syndrome (UARS), sleep apnea, and combinations thereof. Sleep apnea can include OSA, central sleep apnea (CSA), and complex sleep apnea. Reference to “improving” a patient's SDB includes treating, reducing the symptoms of, mitigating, or preventing the SDB. In certain aspects, systems and methods of improving a patient's SDB is preventative as opposed to reactionary in nature. In other words, a system or method of improving a patient's SDB according to certain aspects involves preventing SDB as opposed to detecting an apnea or hypopnea event, for example, and responding to such detected event. By preventing SDB, a treatment system or method can reduce the potential for airway collapse as opposed to reacting to a documented event. A patient suffering from SDB includes a mammal, such as a human being. As used herein with respect to a described element, the terms “a,” “an,” and “the” include at least one or more of the described element unless otherwise indicated. Further, the terms “or” and “and” refer to “and/or” and combinations thereof unless otherwise indicated. As used herein a “patient” includes a mammal such as a human being. The term “machine-learning” can refer to one or more statistical techniques (or algorithms) to progressively improve performance on a specific task without being explicitly programmed. An instruction(s) executable by a processor can be executable by the same processor or multiple processors.

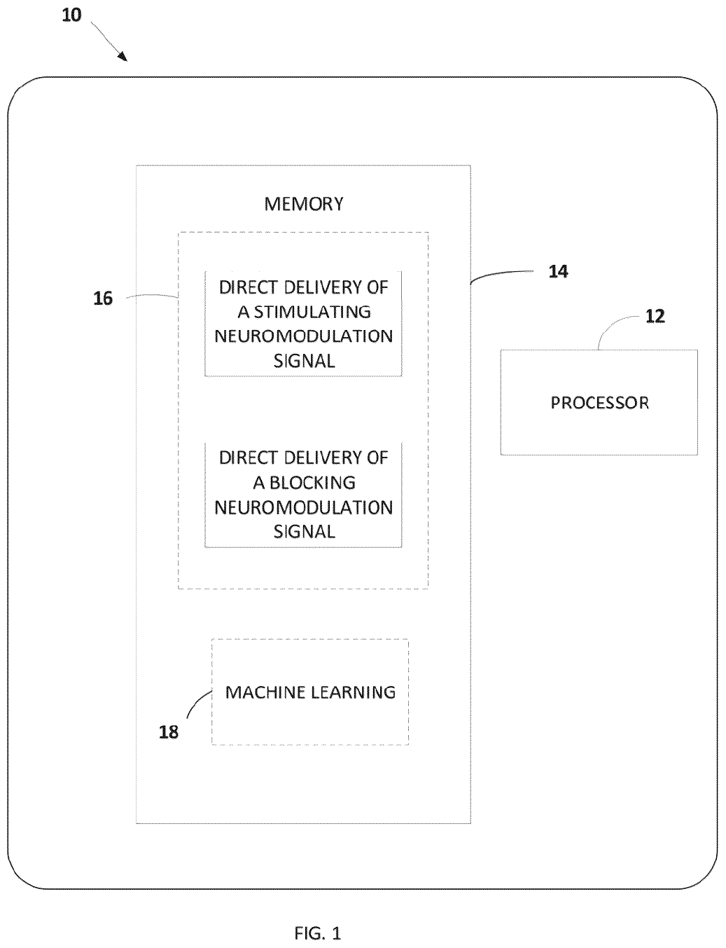

Referring to FIG. 1, in an aspect, a neuromodulation system to improve SBD in a patient having at least one neuromodulation device with at least one electrical contact implanted in or positioned on the patient's body is provided. System 10 can include processor 12 and non-transitory memory 14 storing computer-readable instructions 16 that, when executed by processor 12, cause the at least one neuromodulation device to perform various functions. The computer-readable instructions can include an algorithm for performing the functions and can employ machine learning 18. The instructions can include directing delivery of a stimulating neuromodulation signal to an ansa cervicalis that stimulates activation of the ansa cervicalis innervating one or more infrahyoid muscles. The delivery can be directed, for example, to one or both of the superior root and the inferior root of the ansa cervicalis and can activate efferent/motor fibers to activate the one or more infrahyoid muscles. Without wishing to be bound by a particular mechanism of action, it is believed that activation of infrahyoid muscles (e.g. tightening of these muscles) can reduce upper airway compliance (e.g. stiffen the upper airway) by pulling the thyroid cartilage and/or hyoid bone caudally along with their respective pharyngeal muscle attachments. Upper airway compliance can indicate the potential of the airway to collapse and can be relevant to treating SDB. The processor can direct delivery of the stimulating neuromodulation signal to activate one or more of the infrahyoid muscles including the sternohyoid muscle, the sternothyroid muscle, the omohyoid muscle, the thyrohyoid muscle, or suitable combinations thereof.

The instructions can further include directing delivery of a blocking neuromodulation signal to the ansa cervicalis that blocks activation of efferent fibers that innervate one or more suprahyoid muscles to mitigate undesirable retrograde efferent activation of the one or more suprahyoid muscles that pull the hyoid bone and thyroid cartilage anteriorly and/or superiorly. The delivery can be directed, for example, to the superior root of the ansa cervicalis. In certain aspects, the instructions include directing delivery of a blocking neuromodulation signal to the ansa cervicalis that blocks activation of efferent fibers that innervate the geniohyoid muscle. Mitigating undesirable retrograde efferent activation of the one or more suprahyoid muscles is advantageous because their contraction could dampen or counteract the desired caudal movement of the thyroid cartilage and/or hyoid bone achieved by activation of the infrahyoid muscles

The processor can also execute instructions to direct delivery of a stimulating neuromodulation signal to a hypoglossal nerve that stimulates a genioglossus muscle. In particular, the stimulating neuromodulation signal can activate the efferent/motor fibers of the hypoglossal nerve to activate the genioglossus muscle. Activation of the hypoglossal nerve can cause contraction of desired tongue musculature, thereby reducing or eliminating pharyngeal obstruction generated by posterior collapse of the tongue base by displacing the tongue anteriorly. Such obstruction may not otherwise be treated by ansa cervicalis stimulation alone.

In certain aspects, the processor can execute instructions to direct delivery of a blocking neuromodulation signal to the hypoglossal nerve that blocks activation of efferent fibers that innervate a styloglossus muscle, a hyoglossus muscle, or both. The blocking neuromodulation signal can mitigate undesirable efferent activation of the styloglossus muscle, the hyoglossus muscle, or both. Mitigating undesirable efferent activation of the styloglossus muscle, the hyoglossus muscle, or both can be advantageous because they retract the tongue towards the posterior pharyngeal wall, counteracting the desired anterior displacement.

The processor can also execute instructions to direct delivery of a stimulating neuromodulation signal to the glossopharyngeal nerve that stimulates activation of efferent fibers of the glossopharyngeal nerve. Such efferent fibers can innervate one or more pharyngeal constrictor muscles or a stylopharyngeus muscle to activate the one or more pharyngeal constrictor muscles, the stylopharyngeus muscle, or both. The processor can direct delivery of a stimulating neuromodulation signal to the pharyngeal plexus or a branch thereof, for example. When activated, such one or more pharyngeal constrictor muscles can increase pharyngeal muscle tone to reduce pharyngeal airway collapsibility by stiffening the pharyngeal walls. Stiffening of the pharyngeal walls without complete constriction may render stimulation of the ansa cervicalis and hypoglossal nerve more effective. Regarding the stylopharyngeus muscle, when activated, the stylopharyngeus muscle can move the pharyngeal wall laterally and may increase airway caliber and may also counterbalance a pharyngeal narrowing component of constrictor muscle activation that may occur if muscle activation advances beyond initial pharyngeal wall stiffening.

In certain aspects, the processor can execute instructions to direct delivery of a blocking neuromodulation signal to the glossopharyngeal nerve that blocks activation of afferent fibers of the glossopharyngeal nerve. The blocking neuromodulation signal can mitigate undesirable afferent/sensory effects. Mitigating undesirable afferent activation of is advantageous because afferent activation to central nervous system receptors may lead to neurologic arousal from sleep or may cause undesirable muscle activation through afferent/efferent reflex arcs, such as activation of the pharyngeal gag reflex.

In certain aspects, the processor can execute instructions to direct delivery of a stimulating neuromodulation signal to a neural site, such as a pharyngeal nerve plexus or branch thereof, that stimulates activation of a palatoglossus muscle, a palatopharyngeus muscle, or both. Electrical stimulation of a neural site that innervates the palatoglossus muscle and/or palatopharyngeus muscle during sleep can dilate the retropalatal space and therefore open the patient's upper airway without causing arousal from sleep. Such a system can be utilized in patients with isolated palatal collapse or in conjunction with hypoglossal nerve stimulation, ansa cervicalis stimulation phrenic nerve stimulation, and other neuromodulation systems as part of multi-level airway therapy for SBD, such as OSA.

In certain aspects, the processor can execute instructions to direct delivery of a stimulating neuromodulation signal to the phrenic nerve to stimulate activation of the phrenic nerve to activate the diaphragm. Phrenic nerve stimulation can affect upper airway collapsibility or can be used to treat central sleep apnea. Stimulating the phrenic nerve in isolation can cause airway collapse instead of protecting against it, as diaphragm descent can intrinsically generate a negative pressure gradient within the pharyngeal lumen that can cause collapse before the stabilizing effects are realized. Stimulating both the ansa cervicalis and the phrenic nerve can provide the ability to control airway collapsibility to a greater degree than either one in isolation by enabling individualized control of caudal traction and thoracic expansion through the respiratory cycle.

In certain aspects, the processor can execute instructions to direct delivery of any suitable combination of stimulating neuromodulation signals and/or blocking signals as described above with suitable neuromodulation parameters. Non-limiting examples of neuromodulation parameters include electrical contact selection such as which electrical contact(s) of the neuromodulation device provides a stimulating neuromodulation signal and which electrical(s) contacts provide a blocking neuromodulation signal, stimulation patterns, signal pulse waveform, signal pulse width, signal pulse frequency, signal pulse phase, signal pulse polarity, signal pulse amplitude, signal pulse intensity, signal pulse duration, duty cycle, and combinations thereof. This process can continue until a therapeutic effect is reached with minimal side effects.

The process can also be aided by machine learning 18. The machine learning 18 can include an algorithm that can be trained to recognize certain effects from delivery of the neuromodulation signals. The machine learning 18 can allow active adjustment of neuromodulation parameters of the neuromodulation signals to deliver a therapeutic effect with minimal side effects. The machine learning can employ one or more machine learning algorithms, such as, for example, Decision tree learning, Association rule learning, Artificial neural networks, Deep learning, Inductive logic programming, Support vector machines, Clustering, Bayesian networks, Reinforcement learning, Representation learning, Similarity and metric learning, Sparse dictionary learning, Genetic algorithms, Rule-based machine learning, Learning classifier systems, Feature selection, or the like.

Regarding specific details of a system as provided herein, the processor can comprise one or more microprocessors under the control of a suitable software program. The processor can control various neuromodulation parameters of the neuromodulation device such as, for example, stimulation patterns, electrical contact selection, signal pulse waveform, signal pulse width, signal pulse frequency, signal pulse phase, signal pulse polarity, signal pulse amplitude, signal pulse intensity, signal pulse duration, duty cycle, and combinations thereof. The processor can be programmed to convey a variety of currents and voltages to the electrical contacts and thereby modulate the activity of a neural target site such as a nerve, neuron or nerve fiber. The processor may be programmed to control numerous electrical contacts independently or in various combinations as needed to provide neuromodulation therapy.

An electrical neuromodulation signal can be constant, intermittent, varying and/or modulated with respect to the current, voltage, pulse width, waveform, duty cycle, frequency, amplitude, and so forth. The waveform can be a sine wave, a square wave, a triangular wave, or the like. The type of neuromodulation may vary and involve different waveforms. Optimal stimulation patterns may require a delay in activating one electrical contact before activating another electrical contact or in another coordinated fashion to improve the patient's SBD, whether that involves simultaneous activation or staggered activation of electrical contacts in a coordinated, adjustable fashion. In the case of a stimulating signal, the signal can have, for example, an amplitude ranging from about 0.1 mA to about 5 mA, a pulse width between about 30 μs to about 250 μs, a pulse frequency between about 30 to about 50 Hz, a train length between about 0.1 to about 5 s, and a train interval encoded as either time ranging from about 0.1 to about 5 s, or as a percentage of the encoded train length. In the case of a blocking signal, the signal can have, for example, an amplitude ranging from about 0.1 mA to about 5 mA, a pulse width between about 100 μs to 1000 μs, a frequency ranging from about 1 Hz to about 60 kHz, and a time parameter between 1 and 250 μs to synchronize the blocking signal with the appropriate phase of a complimentary stimulation signal being simultaneously delivered to the same target nerve.

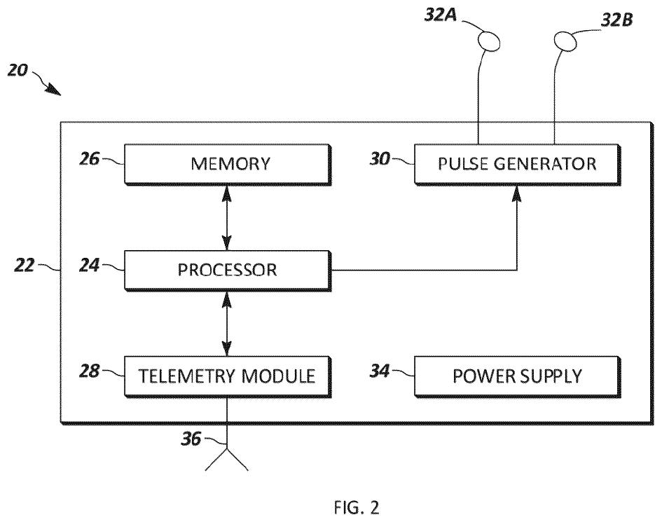

The neuromodulation system can include electronic circuitry, such as one or more electronic circuits for delivering neuromodulation signals enclosed in a sealed housing and coupled to neuromodulation devices, such as cuff electrodes, electrical leads, or neuromodulation devices with other form factors having electrical contacts. FIG. 2 is a block diagram of a neuromodulation system 20 according to an aspect of the present disclosure. Neuromodulation system 20 can include a housing 22 enclosing a processor 24 and associated memory 26, a telemetry module 28, and a pulse generator 30 in electrical communication with electrical contacts 32A and 32B of neuromodulation device(s). Neuromodulation system 20 can also include a power supply 34.

The neuromodulation device can have different form factors such as, for example, an injectable microstimulator, a nerve cuff electrode, a cylindrical lead, a paddle lead, or a transcutaneous patch. Further, as stated above, multiple target sites can be stimulated by the same neuromodulation device or a single target site can be stimulated by a single neuromodulation device. The neuromodulation can be unilateral neuromodulation as well as bilateral neuromodulation of these neural target sites.

Electrical contacts 32 of a neuromodulation device can be located along an exterior surface of housing 22 and can be coupled to pulse generator 30 via insulated feedthroughs or other connections. In other embodiments, electrical contacts 32 can be carried by a neuromodulation device that is a lead or insulated tether electrically coupled to the processor via appropriate insulated feedthroughs or other electrical connections crossing the sealed housing. In still other embodiments, electrical contacts can be incorporated in the housing with externally exposed surfaces adapted to be operably positioned in proximity to a target site proximate to a neural target site and electrically coupled to the processor. The electrical contacts can be controllable to provide electrical signals that may be varied, for example, in voltage, frequency, amplitude, waveform, pulse-width, current, intensity, duty cycle, polarity, duration, and combinations thereof. The electrical contacts can also provide both positive and negative current flow from the electrical contact or can be capable of stopping current flow from the electrical contact or changing the direction of current flow from the electrical contact.

As stated above, a neuromodulation device, such as, for example, a nerve cuff electrode can be placed on the same or different target sites. For example, if the target sites include two separate nerves or nerve segment, a separate nerve cuff electrode or other neuromodulation device can be placed on each nerve or nerve segment with each nerve cuff electrode having its own cathode and anode but connected to the same processor or separate nerve cuff electrodes connected to the same processor but one nerve cuff electrode serving as the cathode and the other serving as the anode, where the electrical field generated captures both nerves or nerve segments. In certain embodiments, a neuromodulation device configured to stimulate a nerve or nerve segment can be combined with a neuromodulation device configured to stimulate another nerve or nerve segment. Still alternatively, a neuromodulation device configured to stimulate a nerve or nerve segment can be part of a device separate from a neuromodulation device configured to stimulate another nerve or nerve segment.

The processor can include any one or more of a microprocessor, a digital signal processor (DSP), an application specific integrated circuit (ASIC), a field-programmable gate array (FPGA), or equivalent discrete or integrated logic circuitry. In some examples, the processor can include multiple components, such as any combination of one or more microprocessors, one or more controllers, one or more DSPs, one or more ASICs, or one or more FPGAs, as well as other discrete or integrated logic circuitry. The functions attributed to the processor herein can be embodied as software, firmware, hardware or any combination thereof. The processor can be programmed to deliver electrical signals having various characteristics. As stated above, the electrical signal may be constant, intermittent, varying or modulated with respect to the current, voltage, pulse-width, waveform, cycle, frequency, amplitude, and so forth. The waveform can be a sine wave, a square wave, or the like. The type of stimulation may vary and involve different waveforms. Optimal activation patterns may require a delay in one electrode before activating another or in another coordinated fashion to optimally open the airway, whether that involves simultaneous activation or staggered activation in a coordinated, adjustable fashion.

Non-limiting examples of a blocking neuromodulation signal include direct current (DC) block, kilohertz frequency alternating current block (KHFACb), anodal block, collision block, quasi-trapezoidal stimulation, and low frequency alternating current block (LFACb). In the case of a DC block, a ramp or DC current is passed through a blocking electrical contact. However, this approach has been associated with the potential for generation of toxic electrochemical byproducts that could damage potentially a nerve. Alternatively or in addition, KHFACb is a method using a sinusoidal charged balanced waveform in the frequency range from about 1 kHz to about 40 kHz that can be delivered. Since the waveform is zero mean with short cycle durations, the charge reverses itself and there is no net accumulated charge to avoid the possibility of nerve damage. To mitigate the effects from a DC block and KHFACb, a blocking waveform can be generated that is a combination of the two blocks. Charged balanced direct current (CBDC) carousal block is a method that mitigates the onset activation where a ramped DC pulse or trapezoidal pulse is used to achieve a block, which is charge balanced with a long but equal charge discharging phase. In addition, high capacitance materials (e.g. Pt black) can be used to prevent reactive species from being produced. LFACb is a pure tone sinusoidal waveform modification of KHFACb differentiated by its amplitude and frequency. Phasic blocking of action potentials can be achieved by reducing the frequency of the sinusoidal waveform by less than approximately 10 Hz, and with less overall current delivery than that required for KHFACb.

The processor may be programmed to control numerous electrical contacts independently or in various combinations as needed to provide neuromodulation. In one example, a neuromodulation therapy protocol to improve an SDB in a patient can be stored or encoded as instructions in memory that are executed by the processor to cause a pulse generator to deliver the therapy via electrical contacts according to the programmed protocol. As such, a neurostimulation device can be pre-programmed with desired stimulation parameters. Although the processor is illustrated in FIG. 2 as being internal to the neuromodulation device, it alternatively can be an external controller such that neuromodulation parameters are remotely modulated to desired settings.

Memory 26 can include computer-readable instructions that, when executed by processor 24, cause the neuromodulation device(s) to perform various functions attributed throughout this disclosure to the neuromodulation device(s). The computer-readable instructions can be encoded within memory 26. The memory can comprise non-transitory computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media with the sole exception being a transitory, propagating signal.

Telemetry module 28 and associated antenna 36 can be provided for establishing bidirectional communication with an external device including, for example, a patient programmer and/or a physician programmer. Examples of communication techniques used by the neuromodulation system and an external device include low frequency or radiofrequency (RF) telemetry, which can be an RF link established via Bluetooth, WiFi, or MICS, for example. Antenna 36 can be located within, along or extend externally from housing 22.

Power supply 46 can be a battery or other power source. The battery can be rechargeable by inductive coupling. The power supply can be inside a neuromodulation device (as illustrated in FIG. 2), at a remote site in or on the patient's body, or away from the patient's body in a remote location. When located away from the body, the neurostimulation device may be powered by bringing a power source external to the patient's body into contact with the patient's skin or at a site remote from the patient's body (e.g. where the electrical energy is delivered through another medium first). When the neuromodulation device is configured as an externally powered device, the power supply can be worn by the patient during sleep to provide power needed to generate stimulation pulses or can be adjacent to the patient (e.g. such as one the patient's bed, under the patient's pillow, one the patient's nightstand, etc.). For example, the power supply can be a battery-powered device including a primary coil used to inductively transmit power to a secondary coil included in the neuromodulation device. The power supply can include one or more primary or rechargeable cells and therefore can include a power adaptor and plug for re-charging in a standard 110V or 220V wall outlet, for example. In some embodiments, the functionality required for transmitting power to the neuromodulation device when the neuromodulation device is embodied as a rechargeable or externally powered device and for programming the neuromodulation device for controlling therapy delivery can be implemented in a single external device.

The neuromodulation system can include other components such as an analog front end or analog-to-digital converter, a multiplexer and other components.

In another aspect, a neuromodulation system can include one or more sensors (not shown) to permit open-or closed-loop control. In an open-loop system, for example, the system can include one or more sensors such that a patient can manage (e.g., prophylactically) improvement of the SDB based on feedback (e.g., detected signals) from the sensor(s). Such detected signals can be indicative of the onset of the SDB, such as changes in muscle or nerve electrical activity, tongue position, oropharyngeal airflow, etc. Upon noticing the signal(s), the patient can then trigger or activate the neuromodulation device to prevent or mitigate the SDB.

In another aspect, the neuromodulation system can include one or more sensors to permit closed-loop control by, for example, automatically responding (e.g., by activation of the neuromodulation device) in response to a sensed physiological parameter, or a related symptom or sign, indicative of the extent or presence of the SDB. Physiological parameters include changes in muscle or nerve electrical activity, tongue position, changes in heart rate or blood pressure, pressure changes in response to respiratory effort, oropharyngeal airflow, etc. Sensors used as part of a closed-or open-loop system can be placed at any appropriate anatomical location on a patient, including a skin surface, an oral cavity, a nasal cavity, a mucosal surface, or at a subcutaneous location.

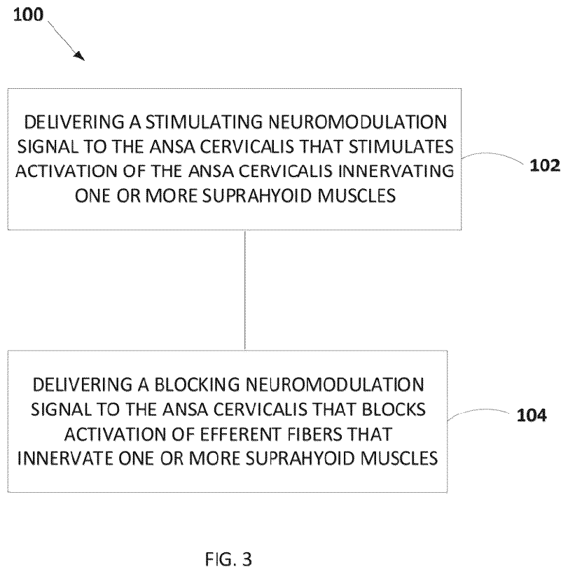

Referring to FIG. 3, in an aspect, a method 100 of treating SDB in a patient suffering therefrom comprises delivering a stimulating neuromodulation signal to an ansa cervicalis that stimulates activation of the ansa cervicalis innervating one or more infrahyoid muscles 102 and delivering a blocking neuromodulation signal to the ansa cervicalis that blocks activation of efferent fibers that innervate one or more suprahyoid muscles 104. The blocking neuromodulation signal can mitigate undesirable retrograde efferent activation of the one or more suprahyoid muscles, including, for example, the geniohyoid muscle.

Regarding the stimulating neuromodulation signal, such a signal can be delivered to stimulate activation of the motor fibers of the ansa cervicalis to activate, for example, the sternothyroid muscle. A stimulating neuromodulation signal can also be delivered to the ansa cervicalis innervating the superior belly of the sternohyoid muscle and/or the inferior belly of the sternohyoid muscle to activate part or all of the sternohyoid muscle. For example, an exemplary target site for delivering a stimulating neuromodulation signal can be the superior root of the ansa cervicalis or proximate to or at the branch point of the superior root innervating the sternohyoid muscle such that the sternohyoid muscle is activated as well as the sternothyroid muscle. In certain aspects a stimulating neuromodulation signal can be delivered to the superior root of the ansa cervicalis or proximate to the superior root to activate part or all of the omohyoid muscle.

In certain aspects, a stimulating neuromodulation signal can be delivered to a target site proximate the ansa cervicalis (e.g. proximate to the inferior root of the ansa cervicalis) also innervating the sternothyroid muscle, the sternohyoid muscle, and omohyoid muscle to activate one or more of the innervated muscles. In certain aspects, a stimulating neuromodulation signal can be delivered simultaneously to the ansa cervicalis in order to stimulate nerve branches from both the superior root and inferior root of the ansa cervicalis innervating the sternothyroid muscle as well as the sternohyoid muscle and omohyoid muscle. In certain aspects, delivering a stimulating neuromodulation signal to a target site (e.g. proximate to or at the branch point of the common trunk nerve or nerves arising from the loop of the ansa cervicalis combining nerve fibers from the superior root and the inferior root and supplying at least the sternothyroid muscle and variably the sternohyoid muscle and omohyoid muscle) can activate at least the sternothyroid muscle and in certain aspects, the sternohyoid muscle and in certain aspects the omohyoid muscle. In certain aspects, delivering a stimulating neuromodulation signal to a target site (e.g. proximate to or at the branch point of the sternothyroid muscle nerve or nerves from the common trunk of the ansa cervicalis) can activate the sternothyroid muscle. The branches to the sternothyroid muscle can be a single nerve fiber or several closely located nerve fibers traveling together. It should be noted that the above target sites are only exemplary and a neuromodulation device can be placed at other parts of the ansa cervicalis including branches thereof. Further, stimulation can be applied to any combination of the above-described sites and branches. For example, a neurostimulation device can be placed proximal or distal to the branch to the omohyoid muscle such that stimulation is capturing only the sternothyroid/sternohyoid fibers. As another example, a cuff electrode or electrodes could surround a single fiber or multiple fibers innervating the sternothyroid muscle.

Regarding the blocking neuromodulation signal, such a signal can be delivered to the superior root of the ansa cervicalis, for example, to reduce or eliminate retrograde motor efferent activation of suprahyoid muscular targets when only activation of infrahyoid musculature is desired.

In certain aspects, a method of improving SDB comprises additionally delivering an electrical signal to a target site proximate to a hypoglossal nerve innervating the genioglossus muscle to activate the genioglossus muscle. A target site can be proximate to the hypoglossal nerve such that delivering an electrical signal activates the motor fibers of the hypoglossal nerve to activate the genioglossus muscle. In certain aspects, an electrical signal is not delivered to the hypoglossal nerve proximal to its branch point as it is believed that separate neuromodulation devices may be needed to potentially provide different strength or timing of stimulation to the ansa cervicalis and the hypoglossal nerve. In other aspects, the hypoglossal nerve can be stimulated proximal or distal to the branch point of the retrusor muscle branches to the styloglossus muscle and/or the hyoglossus muscle. Activation of the hypoglossal nerve can stiffen tongue musculature, reducing or eliminating pharyngeal obstruction generated by posterior collapse of the tongue base that may not otherwise be treated by ansa cervicalis, for example.

In certain aspects, a method further includes delivering a blocking neuromodulation signal to the hypoglossal nerve that blocks activation of efferent fibers that innervate a styloglossus muscle, a hyoglossus muscle or both. Such a blocking neuromodulation signal can avoid undesirable efferent activation of the styloglossus muscle, the hyoglossus muscle or both. Blocking of undesirable motor efferents may be desired when an upstream electrical contact activates multiple efferent motor targets.

In certain aspects, a method of improving SDB comprises additionally delivering a stimulating neuromodulation signal to an efferent fiber of the glossopharyngeal nerve innervating one or more pharyngeal constrictor muscles or a stylopharyngeus muscle to activate the one or more pharyngeal constrictor muscles or the stylopharyngeus muscle. The neural target site can comprise the pharyngeal plexus or a branch thereof, for example. When activated, such one or more pharyngeal constrictor muscles can increase pharyngeal muscle tone to reduce pharyngeal airway collapsibility. When activated, a stylopharyngeus muscle can move the pharyngeal wall laterally.

The side walls of the pharynx are constructed from the pharyngeal constrictors, which are innervated by the pharyngeal plexus that include fibers from cranial nerves IX and X. The nerves that innervate these muscles form a plexus over the outside surface of the pharyngeal constrictor muscles and then penetrate the pharyngeal constrictor muscles to reach the palatoglossus and palatopharyngeus muscles. The motor branches of cranial nerve IX may be responsible for respiratory control of the constrictor muscles and may be identified in the region of the stylopharyngeus muscle. Increased constrictor muscle tone during respiration can reduce pharyngeal collapsibility by stiffening the pharyngeal walls. Stiffening of the pharyngeal walls without complete constriction may render stimulation of the ansa cervicalis and hypoglossal nerve more effective. Stimulation of the stylopharyngeus muscle may increase airway caliber by moving the pharyngeal wall laterally and may also counterbalance a pharyngeal narrowing component of constrictor muscle activation that may occur if muscle activation advances beyond initial pharyngeal wall stiffening.

Stimulating an efferent fiber of the glossopharyngeal nerve innervating one or more pharyngeal constrictor muscles or a stylopharyngeus muscle can be combined with stimulation of the other neural target sites as described herein. Stimulation of the ansa cervicalis may anchor the inferior end of the pharynx by preventing upward movement of the thyroid cartilage and hyoid bone, which may allow contraction of the pharyngeal constrictor muscles and palatopharyngeus muscle to work against a solid anchor as opposed to a mobile insertion point, increasing the effectiveness of co-stimulation of the ansa cervicalis with stimulation of an efferent fiber of the glossopharyngeal nerve or stimulation of the palatopharyngeus muscle.

In certain embodiments, a method includes delivering a neuromodulation signal to efferent fibers of the glossopharyngeal nerve independent of sensory or input signals detected or sensed regarding the neuromuscular state of the airway. For example, an electrical signal can be delivered on a tonic basis or on a duty cycle independent of sensory or input signals detected or sensed regarding the neuromuscular state of the airway. In other words, even though sensory or input signals regarding the neuromuscular state of the airway can be measured, such sensory information may not dictate the stimulation parameters of the electrical signal delivered to the target site in certain embodiments. In certain aspects, an electrical signal is not delivered to sensory fibers that innervate the mucosal layers of the pharynx wall.

In certain aspects, a method includes delivering a blocking neuromodulation signal to the glossopharyngeal nerve to block activation of afferent fibers of the glossopharyngeal nerve. The blocking neuromodulation signal can avoid undesirable sensory effects. The blocking neuromodulation signal can be delivered upstream of the main glossopharyngeal nerve, for example.

In certain aspects, a method of improving SDB comprises additionally delivering a neuromodulation signal to a nerve that innervates a palatal muscle, such as the palatoglossus muscle, the palatopharyngeus muscle, or both to improve SBD in a patient suffering therefrom. The palatoglossus muscle and palatopharyngeus muscle are muscles of the soft palate (also referred to as “palatal muscles”). The palatoglossus muscle originates from the palatine aponeurosis at the posterior part of the hard palate. It descends inferolaterally to insert into the posterolateral surface of the tongue. During its course through the posterior part of the oral cavity, it is covered medially by a mucous membrane, so forming the palatoglossus arch. The palatoglossus muscle functions to close off the oral cavity from the oropharynx by elevating the posterior tongue and drawing the soft palate inferiorly. This muscle is innervated by a branch of the pharyngeal plexus, which functions independently of the hypoglossal nerve and the rest of the intrinsic and extrinsic tongue musculature. The palatopharyngeus muscle forms the palatopharyngeal arch. It attaches superiorly to the hard palate and palatine aponeurosis and inferiorly to the lateral wall of the pharynx and the thyroid cartilage. It functions to tense the soft palate and pull the pharyngeal walls superiorly, anteriorly, and medially during swallowing, effectively closing off the nasopharynx from the oropharynx.

Such methodology of delivering an electrical signal to a nerve innervating a palatal muscle to improve SDB is different than indirect methods of muscle stimulation such as transcutaneous electrical pacing, which have been found to be inconsistent and poorly tolerated by sleeping patients. Direct intramuscular stimulation via fine wire electrode placement or other techniques would be impractical for daily use as it would require daily uncomfortable piercing of the skin or lining of the mouth to access muscle tissue. Electrical stimulation of a nerve that innervates the palatoglossus muscle and/or palatopharyngeus muscle during sleep can dilate the retropalatal space and therefore open the patient's upper airway without causing arousal from sleep. Such a method can be utilized in patients with isolated palatal collapse or in conjunction with hypoglossal nerve stimulation, ansa cervicalis stimulation and/or phrenic nerve stimulation as part of multi-level airway therapy for SBD, such as OSA.

In certain embodiments, the target site of a nerve that innervates the palatoglossus muscle and/or the palatopharyngeus muscle is the pharyngeal plexus or a branch thereof. Preferably, the electrical signal delivered to a target site of a nerve that innervates the palatoglossus muscle and/or the palatopharyngeus muscle stimulates motor fibers of the pharyngeal plexus that innervate the patient's soft palate.

In certain embodiments, a target site for stimulation is not a cranial root of an accessory nerve of the patient as targeting the cranial root of the accessory nerve or the root of the vagus nerve would result in diffuse, non-specific stimulation. For example, activation of the vagal root could simultaneously activate the levator veli palatini muscle, which opposes the action of the palatoglossus and palatopharyngeus muscles. Stimulation of the cranial root of the spinal accessory nerve could similarly cause non-specific activation of the pharyngeal plexus musculature. Moreover, the cranial root of the accessory nerve is not known to join the vagus nerve in all patients. If the cranial root of the accessory nerve did not join the pharyngeal plexus it would instead remain with the spinal root of the accessory nerve. Stimulation of the cranial root of the accessory nerve would therefore cause unintentional stimulation of the spinal root of the accessory nerve, which would cause undesirable activation of the sternocleidomastoideo trapezius muscles.

In certain aspects, a method of improving SDB comprises stimulating the ansa cervicalis to activate one or more infrahyoid strap muscles in combination with stimulating the phrenic nerve to activate the diaphragm. Phrenic nerve stimulation can affect upper airway collapsibility. Stimulating the phrenic nerve in isolation can cause airway collapse instead of protecting against it, as diaphragm descent intrinsically generates a negative pressure gradient within the pharyngeal lumen that can cause collapse before the stabilizing effects are realized. Stimulating both the ansa cervicalis and the phrenic nerve can provide the ability to control airway collapsibility to a greater degree than either one in isolation by enabling individualized control of caudal traction and thoracic expansion through the respiratory cycle.

Delivering a neuromodulation signal to any one or more of the above target sites can be accomplished by placing one or more electrical contacts proximate to the target site. The electrical contacts can be placed proximate to a target site in a variety of different ways, such as, for example, transcutaneously, percutaneously, subcutaneously, intramuscularly, intraluminally, transvascularly, intravascularly, or via direct open surgical implantation. The neuromodulation (stimulating and/or blocking) can be unilateral neuromodulation or bilateral neuromodulation.

In certain aspects, instead of delivering a blocking neuromodulation signal to the above described neural target sites, the intensity of stimulation of a portion of any of these nerves' desired motor targets may be reduced to better balanced the ratio or alter the ratio of recruitment in the desired muscles.

Methods as disclosed herein can be used as part of a closed-loop system (as described in more detail below). Such a method can include sensing a physiological parameter associated with SDB, generating a sensor signal based on the physiological parameter, and activating the electrode(s) to adjust application of the electrical signal to the target site in response to the sensor signal to improve the patient's SDB.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance. Further, while the above is described with respect to electrical stimulation, other forms of energy could be used, such as, for example, ultrasound, magnetic, or optical energy.

What is claimed is:

1. A neuromodulation system to improving sleep disordered breathing comprising:
a processor; and
a non-transitory computer readable medium storing executable instructions executable by the processor to:
direct delivery of a stimulating neuromodulation signal to an ansa cervicalis that stimulates activation of the ansa cervicalis innervating one or more infrahyoid muscles; and
direct delivery of a blocking neuromodulation signal to the ansa cervicalis that blocks activation of efferent fibers that innervate one or more suprahyoid muscles, the blocking neuromodulation signal mitigating undesirable retrograde efferent activation of the one or more suprahyoid muscles.

2. The neuromodulation system of claim 1, further comprising an instruction to:
direct delivery of a stimulating neuromodulation signal to hypoglossal nerve that stimulates activation of a genioglossus muscle.

3. The neuromodulation system of claim 1, further comprising an instruction to:
direct delivery of a blocking neuromodulation signal to a hypoglossal nerve that blocks activation of efferent fibers that innervate a styloglossus muscle, a hyoglossus muscle, or both, the blocking neuromodulation signal mitigating undesirable efferent activation of the styloglossus muscle, the hyoglossus muscle, or both.

4. The neuromodulation system of claim 1, further comprising an instruction to:
direct delivery of a stimulating neuromodulation signal to a glossopharyngeal nerve that stimulates activation of efferent fibers of the glossopharyngeal nerve.

5. The neuromodulation system of claim 1, further comprising an instruction to:
direct delivery of a blocking neuromodulation signal to a glossopharyngeal nerve that blocks activation of afferent fibers of the glossopharyngeal nerve, the blocking neuromodulation signal mitigating undesirable sensory effects.

6. The neuromodulation system of claim 1, further comprising an instruction to:
direct delivery of a stimulating neuromodulation signal to a hypoglossal nerve that stimulates a genioglossus muscle; and
direct delivery of a blocking neuromodulation signal to the hypoglossal nerve that blocks activation of efferent fibers that innervate a styloglossus muscle, a hyoglossus muscle, or both, the blocking neuromodulation signal mitigating undesirable efferent activation of the styloglossus muscle, the hyoglossus muscle, or both.

7. The neuromodulation system of claim 1, further comprising an instruction to:
direct delivery of a stimulating neuromodulation signal to a glossopharyngeal nerve that stimulates activation of efferent fibers of the glossopharyngeal nerve; and
direct delivery of a blocking neuromodulation signal to the glossopharyngeal nerve that blocks activation of afferent fibers of the glossopharyngeal nerve, the blocking neuromodulation signal mitigating undesirable sensory effects.

8. The neuromodulation system of claim 1, further comprising an instruction to:
direct delivery of a stimulating neuromodulation signal to a hypoglossal nerve that stimulates activation of a genioglossus muscle;
direct delivery of a blocking neuromodulation signal to the hypoglossal nerve that blocks activation of efferent fibers that innervate a styloglossus muscle, a hyoglossus muscle, or both, the blocking neuromodulation signal mitigating undesirable efferent activation of the styloglossus muscle, the hyoglossus muscle, or both;
direct delivery of a stimulating neuromodulation signal to a glossopharyngeal nerve that stimulates activation of efferent fibers of the glossopharyngeal nerve; and
direct delivery of a blocking neuromodulation signal to the glossopharyngeal nerve that blocks activation of afferent fibers of the glossopharyngeal nerve, the blocking neuromodulation signal mitigating undesirable sensory effects.

9. The neuromodulation system of claim 1, further comprising an instruction to:
direct delivery of a stimulating neuromodulation signal to a neural site that stimulates activation of a palatoglossus muscle, a palatopharyngeus muscle, or both.

10. The neuromodulation system of claim 1, further comprising an instruction to:
direct delivery of a stimulating neuromodulation signal to the phrenic nerve to stimulate activation of the diaphragm.

11. A method to improve sleep disordered breathing in a patient suffering therefrom comprising:
delivering a stimulating neuromodulation signal to an ansa cervicalis that stimulates activation of the ansa cervicalis innervating one or more infrahyoid muscles; and
delivering a blocking neuromodulation signal to the ansa cervicalis that blocks activation of efferent fibers that innervate one or more suprahyoid muscles, the blocking neuromodulation signal mitigating undesirable retrograde efferent activation of the one or more suprahyoid muscles.

12. The method of claim 11, further comprising:
delivering a stimulating neuromodulation signal to a hypoglossal nerve that stimulates activation of a genioglossus muscle.

13. The method of claim 11, further comprising:
delivering a blocking neuromodulation signal to a hypoglossal nerve that blocks activation of efferent fibers that innervate a styloglossus muscle, a hyoglossus muscle, or both, the blocking neuromodulation signal avoiding undesirable efferent activation of the styloglossus muscle, the hyoglossus muscle, or both.

14. The method of claim 11, further comprising:
delivering a stimulating neuromodulation signal to a glossopharyngeal nerve that stimulates activation of efferent fibers of the glossopharyngeal nerve.

15. The method of claim 11, further comprising:
delivering a blocking neuromodulation signal to the glossopharyngeal nerve that blocks activation of afferent fibers of the glossopharyngeal, the blocking neuromodulation signal avoiding undesirable sensory effects.

16. The method of claim 11, further comprising:
delivering a stimulating neuromodulation signal to a hypoglossal nerve that stimulates activation of a genioglossus muscle; and
delivering a blocking neuromodulation signal to the hypoglossal nerve that blocks activation of efferent fibers that innervate a styloglossus muscle, a hyoglossus muscle, or both, the blocking neuromodulation signal mitigating undesirable efferent activation of the styloglossus muscle, the hyoglossus muscle, or both.

17. The method of claim 11, further comprising:
delivering a stimulating neuromodulation signal to a glossopharyngeal nerve that stimulates activation of efferent fibers of the glossopharyngeal nerve; and
delivering a blocking neuromodulation signal to the glossopharyngeal nerve that blocks activation of afferent fibers of the glossopharyngeal nerve, the blocking neuromodulation signal mitigating undesirable sensory effects.

18. The method of claim 11, further comprising:
delivering a stimulating neuromodulation signal to a hypoglossal nerve that stimulates activation of a genioglossus muscle;
delivering a blocking neuromodulation signal to the hypoglossal nerve that blocks activation of efferent fibers that innervate a styloglossus muscle, a hyoglossus muscle, or both, the blocking neuromodulation signal mitigating undesirable efferent activation of the styloglossus muscle, the hyoglossus muscle, or both;
delivering of a stimulating neuromodulation signal to a glossopharyngeal nerve that stimulates activation of efferent fibers of the glossopharyngeal nerve; and
delivering a blocking neuromodulation signal to the glossopharyngeal nerve that blocks activation of afferent fibers of the glossopharyngeal nerve, the blocking neuromodulation signal mitigating undesirable sensory effects.

19. The method of claim 11, further comprising:
delivering a stimulating neuromodulation signal to a neural site that stimulates activation of a palatoglossus muscle, a palatopharyngeus muscle, or both.

20. The method of claim 11, further comprising:
delivering a stimulating neuromodulation signal to the phrenic nerve to stimulate activation of the diaphragm.
